(19) United States
(12) Patent Application Publication
(10) Pub. No.: US 2009/0247978 A1
(43) Pub. Date: Oct. 1, 2009

(54) ABSORBENT PRODUCT

(75) Inventor: Elisabeth Boissier, Vallda (SE)

Correspondence Address: DRINKER BIDDLE & REATH (DC), 1500 K STREET, N.W., SUITE 1100, WASHINGTON, DC, 20005-1209, US

(73) Assignee: SCA HYGIENE PRODUCTS AB, Goteborg (SE)

(21) Appl. No.: 12/304,250

(22) PCT Filed: Jun. 30, 2006
(86) PCT No.: PCT/SE2006/000814

§ 371 Date: Dec. 10, 2008

(51) Int. Cl. A61F 13/15

(52) U.S. Cl. 604/378

(57) ABSTRACT

An absorbent product having an extent in a plane in the longitudinal direction and the lateral direction and a thickness perpendicular to the plane when the product is positioned in a plane position. The absorbent product includes a backing layer and a top layer, the absorbent product exhibiting in the longitudinal direction a rear section, a front section and positioned between them a central section, and exhibiting in the lateral direction a first lateral zone, a second lateral zone and positioned between them a central zone. The top layer includes slits as the only openings in an area which fully or partially encloses that part of the absorbent product that is formed by the section of the central zone and the central section, and which constitutes the wet area of the product. Lotion is placed directly under the top layer within the area containing slits.

### CROSS-REFERENCE TO PRIOR APPLICATION

This application is a §371 National Stage Application of PCT International Application No. PCT/SE2006/000814 filed Jun. 30, 2006.

### FIELD OF THE INVENTION

The invention relates to an absorbent product having an extent in a plane in the longitudinal direction and the lateral direction and a thickness perpendicular to the plane when the product is positioned in a plane position. The absorbent product includes a backing layer and a top layer. The product comprises in the longitudinal direction a rear section, a front section and between them a central section, and exhibits in the lateral direction a first lateral zone, a second lateral zone and positioned between them a central zone.

### BACKGROUND OF THE INVENTION

In previously disclosed absorbent products, the top layer comprises a layer of material that is liquid-permeable in order to be able to transport liquid from the top layer to the subjacent absorption body. The liquid can be menstrual fluid or urine, and the choice of the material used for the top layer depends on which of these liquids the absorbent product is primarily intended to take up. When choosing the material, consideration must also be given to the required suitability of the top layer having regard for the need for the wearer to experience the material as comfortable against his/her body, and for the wearer to experience a sensation of dryness after the liquid has been excreted. A hydrophobic material is already known to give such a dry sensation. Also previously disclosed is the embodiment of a water-impermeable layer of material with holes in order to make the top layer liquid-permeable, at the same time as the water-impermeable material prevents the transport of liquid from the absorption body in other respects.

The problems associated with the choice of material for the top layer are particularly great in the lateral zones, because the lateral zones of the absorbent product make contact at least partially with a wearer's groin. The nature of the problem is that the groin moves relative to the lateral zones, which can give rise to chafing, and that the lateral zones become crumpled. Movement by the wearer also causes the absorbent product to describe simultaneous movement in the lateral zones, which imposes high demands on the material in order for the material not to rupture. The movement in the lateral zones is usually less than the movement in the central zone, which can give rise to chafing on the wearer if an inappropriate material is used in the lateral zones.

WO 03/015681 depicts an absorbent product comprising a top layer of a film or nonwoven material containing a number of holes, the holes being larger in the central zone than in the lateral zones. The small holes in the lateral zones are provided to give the material a softer feel compared with a non-perforated material. One problem associated with holes is that the holes are always open and permit excessive reflux and leakage of material lying under the top layer. The problems mentioned above are especially great in the lateral zones of the absorbent product, because movement in the lateral zones influences the absorption body in such a way that the subjacent material and fluid exhibit a tendency to be transported out through the holes by the movement in the lateral zone. Making the holes so small that the subjacent material is not able to leak out through the holes, or so small that the moisture reflux problem is reduced, would reduce the admission capacity of the top layer to the subjacent material to an unacceptably low level.

In addition to perforating the top layer in order to make the lateral zones softer, coating the top layer with a lotion with the ability to reduce the risk of chafing in the wearer, and which is also of a hygienic nature for the removal of menstrual fluid and pubic hairs, is previously disclosed in US 2005/0154362, for example. A problem associated with the coating of the top layer with lotion is that the top layer can become sticky, and another problem is that the lotion substance may need to be adapted in order to prevent the top layer from becoming sticky, for example by possessing an essentially solid consistency at a temperature below the temperature of the wearer's lower abdomen.

A wish and a need accordingly remain for an absorbent product, in which the above problems are overcome by means of a top layer having improved characteristics in the lateral zones as far as maximum liquid-permeability, minimal reflux, minimal leakage of loosely lying material, and the transfer of lotion to the wearer's skin is concerned, at the same time as the top layer is provided with a soft and comfortable surface for the wearer when the wearer moves.

### BRIEF SUMMARY OF THE INVENTION

The invention relates to an absorbent product having an extent in a plane in the longitudinal direction and the lateral direction and a thickness perpendicular to the plane when the product is positioned in a plane position. The absorbent product comprises a backing layer and a top layer. The product is theoretically subdivided in the longitudinal direction in such a way that the product exhibits a rear section, a front section and between them a central section. The absorbent product is delimited by a peripheral edge part. The product is theoretically subdivided, from the edge part in the lateral direction, in such a way that the product exhibits a first lateral zone, a second lateral zone and positioned between them a central zone. The invention is characterized in that the top layer comprises slits as the only openings in an area which fully or partially encloses that part of the absorbent product that is formed by the section of the central zone and the central section, and which constitutes a so-called wet area, and in that lotion is placed directly under the top layer within the area containing slits.

The wet area is the area of the absorbent product which is intended to be wetted first by excreted bodily fluid, and which essentially coincides with that part of the absorbent product that is formed by the section of the central zone and the central section, and which can vary in size and position depending on the principal area of application of the absorbent product, for example as a light incontinence pad or sanitary towel.

The subdivision of the absorbent product into zones and sections is of a theoretical nature without distinct boundaries and is made for the sole purpose of facilitating an appreciation of the invention. The laterally and longitudinally extending lines can thus be straight or curved, depending on the shape of the product. The wet area is thus not restricted to having a square or rectangular geometry. It may be mentioned here by way of example that the wet area can have any appropriate geometry, for example circular, oval, triangular, etc. The peripheral part containing slits in accordance with the invention, that is to say the part of the top layer which at least partially encloses the wet area, thus has a corresponding shape at the interface with the wet area. The position of the slits is thus able to follow the contours of the wet area in the lateral zones, the rear section and the front section, and is not restricted to positions within the area described above that is defined by the lateral zones, the rear section and the front section, excluding the section of the central zone and the central section. In a corresponding manner, the lateral zones, the rear section and the front section follow the contours of the absorbent product, that is to say they follow the edge part of the absorbent product.

When using the absorbent product as an incontinence pad, the wet area includes the part of the absorbent product which, when it is being used by a wearer, lies facing towards and in close proximity to the wearer's urethra. The wet area thus constitutes the part of the absorbent product that will receive the largest flow of liquid in conjunction with the excretion of liquid waste by the wearer. In the case of a sanitary towel, the centre of the wet area can be displaced somewhat in relation to the urethra in such a way that the wet area is positioned against that part of a women's lower abdomen from which the flow of menstruation fluid is discharged.

The absorbent product can be a panty liner, an incontinence pad or a sanitary towel, which can be used as undergarment protection in conjunction with menstruation or light incontinence or to protect the undergarments from discharges, etc.

The expression slit is used here to denote an essentially one-dimensional through going opening in a layer of material having an extent in the longitudinal direction and a depth which depends on the thickness of the layer of material and the manner in which the slit is executed in the material. The slit comprises two interfaces, each delimited by the extent of the slit in the longitudinal direction and the thickness of the layer of material. Since the layer of material is unaffected by external forces, the interfaces are in contact with one another and close the opening in the same way as if a slit were not to be present, but with the difference that the bonds which otherwise hold the layer of material together are absent in the section of the layer of material in which the slits are present. The slits are manufactured by rupturing the bonds in the layer of material over the distance required for the extent of the slit. The bonds can be ruptured, for example, by producing an incision in the layer of material by means of slitting, cutting or in some other way. This is entirely true in a theoretical model, although in practical reality material will disappear on a micrometric scale as a consequence of, for example, blunt tools, etc. The loss of material should not be confused with the loss of material when making holes, where the opening is not one-dimensional, but rather two-dimensional, due to the requirement in the case of holes for material to be removed in such a way that a permanent opening is formed, where corresponding interfaces are not able to make contact with one another if the layer of material is not influenced by external forces. In its uninfluenced state, the slit thus exhibits an extent in one direction and has the same thickness as the material.

The definition of slits versus holes is important, because slits offer the advantages that are sought by the present invention. Unlike holes, slits remain closed up to the point in time at which the layer of material is influenced by external forces, when the slits are caused to open by the interfaces being displaced in relation to one another.

This advantage is utilized in the invention in that the slits in the absorbent product before use are essentially closed and are able to retain lotion that has been placed under the top layer in the lateral zones. When the product is used, the slits are influenced by the movement of the wearer in such a way that they open and close, which minimizes the risk of lotion leaking out when the product is in the position of rest, that is to say when it is not being used. Lotion with a density that would give rise to stickiness if it were to be applied to the top layer can thus be used in the present invention without the risk of stickiness, in that it is placed under the top layer and is released via the slits onto the wearer's skin only during use. Because the slits open during use and are closed at other times, the risk of the absorbent product being made sticky by leaking lotion before use is excluded, although lotion is released via the slits during use onto the areas of the wearer's skin that are subject to friction.

According to one embodiment of the invention, the absorbent product comprises an absorption body positioned between the top layer and the backing layer. The absorption body has a first surface and a second surface, the backing layer being arranged over the second surface of the absorption body and the top layer over the first surface. In those cases in which an absorption body is not present in the absorbent product, the backing layer can comprise an absorbent material of the same type as the absorption body. In those cases in which the backing layer lacks an absorbent material, such an absorbent material can be included in the top layer, or the top layer can be regarded as being liquid-absorbent if it is capable of retaining at least a small quantity of liquid. In the latter case, the absorbent product can include, for example, a panty liner intended to protect undergarments from discharges and small quantities of menstrual fluid.

Another advantage of a slit is that the layer of material is breathable and liquid-permeable in precisely the same way as when a hole is made in the material, including if the layer of material without slits is airtight and watertight, but with the difference and the advantage that any liquid that has been absorbed by the subjacent absorption body finds it more difficult to pass through the layer of material because the slits are open and closed alternately, whereas a hole is always open and in this way does not restrict the reflux of liquid from the absorption body.

The slits in the open state possess a larger open area than the area that a circular hole would possess for a given diameter of the hole corresponding to the maximum opening in the slit, because the length of the slit exceeds its width. One advantage of the slit is thus that the opening in the slit is elongated and possesses a smaller maximum opening width than a corresponding radius in a circular hole, which gives good liquid-permeability at the same time as lotion is able to penetrate out through the opening during use. Minimum reflux from the absorption body is also obtained, as well as a soft feeling against the wearer's skin. Furthermore, a larger open area provides better transport of the air through the top layer, that is to say the ability of the top layer to breathe is increased.

The top layer comprises one or a plurality of layers of material and is able, before slitting takes place, to be either liquid-permeable or water-tight, or vapor-tight, or vapor-permeable, or air-tight, or air-permeable, or to have an appropriate combination of the various characteristics. The top layer can include a woven or nonwoven textile material, a plastic material, a fibre material or a combination of the various materials, or some other appropriate material.

The backing layer is appropriately a moisture barrier layer which can be vapor-permeable or vapor-tight. The backing layer can include a treated textile material, or a plastic material, or a fibre material or a combination of these materials.

The top layer can comprise holes or slits, including in the central zone. The central zone is not influenced by the movement of the wearer in the same way as the lateral zones, and entirely different conditions apply there for this reason. Holes can thus be present in the central zone, but not in the lateral zones, for the reasons given above.

The expression movement of the user is used here to denote the movement performed by the wearer in the event of a change in the position of his/her legs, when the inside of the thighs influences the lateral zones of the absorbent product. Such a change in the position of the inside of the thighs relative to the absorbent product occurs in both a sitting, standing, reclining and crouching position, etc., and when the wearer crawls, creeps, walks, runs, jumps, cycles, swims, etc. Even a very small change in position in the lateral zones is sufficient for the slits to be opened and closed in accordance with the invention, for which reason every movement by the wearer in principle gives rise to a movement in the lateral zones.

The absorbent product relates to protection for menstruation and light incontinence. The invention is not intended for diapers or incontinence pads of the panty type, because such absorbent products have a different construction from means of protection such as sanitary towels, panty liners and incontinence pads. The absorbent product in accordance with the invention can essentially be accommodated in a normally sized panty and does not have the front, rear and side panels that are present in diaper pants and which form leg openings and a waist part respectively.

According to a preferred embodiment of the invention, the top layer comprises slits only in the lateral zones, and these can be restricted, furthermore, to only the central section of the lateral zones. The advantages of the slits described above by comparison with holes are especially advantageous in the lateral zones and in the central section of the lateral zones, because the absorbent product is influenced when it is being worn to an especially high degree by the movement of the wearer in these areas. The slits thus open and close at a greater frequency in the lateral zones than in the front section and the rear section, for example. The invention is not restricted to this area, however, but in accordance with another embodiment of the invention the top layer comprises slits in the lateral zones in the rear section and/or in the front section and/or in the central section. The slit areas thus partially enclose the wet area in these embodiments.

According to one embodiment of the invention, the top layer comprises slits in the lateral zones and in the rear section and/or in the front section of the central zone, which means that the top layer comprises slits in an area which entirely surrounds the wet area.

The manner in which the slits extend in the top layer depends on a number of factors, such as the direction of movement of the web of material during the slitting operation and the choice of material for the top layer. It can be mentioned here by way of example that a slit will open when it is subjected to forces that act at an angle towards the direction in which the slit extends. The natural tendency for the slit to open is at its greatest when the forces act upon the slit in a direction oriented at 90° to the direction in which the slit extends. The top layer is manufactured in a web of material having a movement in a machine direction which usually coincides with the longitudinal direction of the absorbent product or its lateral direction. In conjunction with its manufacture, the web of material is influenced by forces in the machine direction which cause slits which lie perpendicular to the machine direction to be influenced to a maximum extent by these forces. The forces involved in this case can cause the material to split at the slits or, at any rate, can cause the slits to open essentially permanently. What is more, the finished absorbent product will contain slits having an extent either in the longitudinal direction or in the lateral direction, which will mean that the slits are affected essentially only by forces acting in one direction. If the slits are instead oriented at an angle to the machine direction, there is a smaller risk of the top layer splitting during manufacture. From the point of view of the product, a significant advantage is achieved in that a shape and an extent are imparted to the slits that are affected by forces both from the lateral direction and from the longitudinal direction and at angles in between. The comparisons indicated above apply to a slit with a given length. The fact that the slits are affected by forces in the lateral direction and in the longitudinal direction, and at angles in between, means that the natural tendency of the slits to open and close as the wearer moves will increase, because movement by the wearer will give rise to forces both in the lateral direction and in the longitudinal direction and in directions in between.

The slits themselves can be straight, S-shaped, V-shaped, Z-shaped, U-shaped, or can possess any other suitable shape. The slits can also comprise combinations of different shapes, for example a plurality of straight or curved slits arranged consecutively and having the same or a different length, where every other slit is oriented at an angle (preferably essentially 90°) in relation to the essential longitudinal extent of the preceding slit, but where the slits are situated at a distance from one another. The slits are thus arranged at an angle of between 0 and 180° relative to a longitudinally extending centre line, preferably in the range from 20°-65° and/or 110°-155° in relation to the longitudinally extending centre line. Different parts of the slits can have a different direction in relation to the centre line.

In order to describe the slit in more detail, the following description is based on a top layer having an extent in a single plane. It must be pointed out, however, that the absorbent product, when it is being worn, has a three-dimensional extent that is adapted in accordance with the wearer's body. The slit can be made in the top layer by means of a through going incision in the top layer perpendicular to the plane surface, but it can also be made in the top layer by means of a through going incision at an angle to the plane surface. In the latter case, the opposing edge parts of the slit overlap one another and change the characteristics of the slit in such a way that the top layer is air-permeable and liquid-permeable as a result of the overlapping parts being raised at an angle towards the plane surface, at the same time as a material lying loosely under the top layer finds it more difficult to escape through the slit than in the case of a slit with a straight incision, because the loose material is obliged to make its way through a longer channel than in the case of a straight incision. Furthermore, the overlapping parts permit air and water to diffuse out through the closed slit, at the same time as the material lying loosely on the under side of the top layer is prevented from finding its way out because it is too large to be able to diffuse out. A further advantage of a slit with inclined opposing edges is that a small lateral displacement, that is to say in the direction in which the plane surface extends, does not cause the slit to open, because the overlapping parts cover one another for the entire length of the opposing edge parts, in conjunction with which the material lying loosely is also restrained in the event of a quite small movement in the material. The top layer is relatively thin, for which reason the maximum movement of the lateral displacement must be small, although the advantages indicated above are also available when the lateral displacement exceeds the maximum movement, because the slit is caused to open less than in the case of a straight incision. In the case of a straight slit, however, air, water vapor and, where appropriate, water can also diffuse out through an opened slit before the slit has been widened to such an extent that the loose material is able to find its way out.

The absorption body is appropriately manufactured from a suitable fibre material in the form of natural or synthetic fibres having absorbent properties, or a mixture of natural fibres and synthetic fibres or other absorbent materials of a previously disclosed kind that are suitable for use in sanitary towels, incontinence pads and panty liners, for example. The absorption body can also contain a predetermined proportion, for example 20-60%, of superabsorbent materials, that is to say polymer materials in the form of particles, fibres, flakes or similar, which have the capacity to absorb and to chemically bind liquid equivalent to several times their own weight while forming an aqueous gel. This provides a very high water-absorbent capacity in the finished product.

The absorption body can exhibit different forms, for example an essentially elongated and rectangular form, or alternatively some other more irregular form, for example hourglass or triangular form.

The liquid-permeable top layer preferably includes one or more layers of one or more of the following materials: a fibrous material, for example a soft nonwoven material, plastic film, mesh, open-celled foam, material laminate, etc. The top layer is preferably fully or partially perforated, that is to say slits are made in the top layer in accordance with the above, and holes can be present in the wet area. The top layer can appropriately include a perforated plastic film, for example a thermoplastic plastic material such as polyethylene or polypropylene, or a mesh-like layer of synthetic or textile material. Synthetic mono-, bi-, or multi-component fibres, made of polymers such as polyethylene, polypropylene, polyester, nylon or the like, are preferably used as a nonwoven material. Mixtures of different types of fibres can also be used for the aforementioned nonwoven material. The invention is not, however, restricted in principle to use only for top layers which include nonwoven material, but can also be applied in conjunction with the processing of other materials, for example films made of thermoplastics such as polyethylene or polypropylene.

The invention can also be implemented with a top layer which includes different types of laminates or combinations of laminates and/or single layers. For example, the top layer can include a number of different laminates or single layers which cover parts of the surface of the product. In the event that the product includes a plurality of laminates or single layers, for example subdivided into a plurality of longitudinal parts having different sections, these different sections can include different materials and can possess different characteristics. For example, each section can then have different types of perforation, hole positioning, dimensions, hydrophobicity, etc. The different sections can be joined together by means of ultrasonic welding in a previously disclosed manner that is not described here in detail.

The liquid-permeable top layer is preferably manufactured from a material that exhibits characteristics such as dryness and softness during the time when the absorbent product is being worn, because this top layer is in contact with the wearer's body. It is also desirable for the top layer to have a soft and textile-like surface which remains dry, even in the event of repeated wetting. The top layer can include a nonwoven material, for example, with a soft and smooth surface, such as a spunbond material made from polypropylene fibres. A perforated, hydrophobic nonwoven material may be used in order to permit the surface that is closest to the wearer's body to be kept dry, in conjunction with which holes are formed in the material that are larger than the distance between the fibres in the material. In this way, liquid can be led down through the holes in the top layer to the subjacent absorption body. Other examples of materials for the top layer are perforated plastic films such as a perforated polyester film. The top layer can be joined together with the subjacent backing layer and the absorption body, for example by means of adhesive, ultrasonic jointing or by means of some form of thermal bonding.

The top layer can also be a three-dimensional laminate of nonwoven and plastic film or a carded, thermally bonded material based 100% on polypropylene. The plastic film can be hydrophilic, pre-perforated (with small holes) and manufactured from a mixture of polyethylene and polypropylene. The nonwoven materials can have a weight per unit area in the range from 12-100 gsm, and in particular in the range from 15-60 gsm.

The nonwoven part of the top layer can also be a spunbond nonwoven material, an air-thru nonwoven material, a spunlace nonwoven (hydroentangled) material, a meltblown nonwoven material, or a combination of these. The raw material can be polypropylene (PP), polyethylene (PE) polyester (PET), polyamide (PA), or a combination of these. If a combination is used, this can be a mixture of fibres from different polymers, although each fibre can also contain different polymers (for example PP/PE bi-component fibres or PP/PE copolymers). Where appropriate, the plastic film can include PE or PP, PET, PLA or amyl (or any other thermoplastic polymer), or a mixture or copolymers of the aforementioned polymers.

The perforated top layer can also be manufactured from a single layer of material, such as a nonwoven material or a film (as described above).

The holes in the top layer can be oval and slightly elongated in the direction of the machine. The holes can be round/circular or oval in the direction of the machine or the transverse direction. The holes in the wet area can also be replaced by slits, which by definition differ from the holes in that the slits do not constitute constant openings, but instead are through going incisions in the layer of material. The slits are opened and closed by movement in the material.

According to the invention, the slits are from 2 mm up to 15 mm in length, and preferably lie in the range from 3-10 mm. The length of the slits is measured along the boundary surfaces of the slits in a direction essentially perpendicular to the thickness of the top layer and when the slit is in its closed state.

The slits are arranged in the top layer with a mutual distance between the slits having a size in the order of 5-15 mm, although this is dependent on a range of factors, for which reason the distance between the slits can vary depending, among other things, on the material in the top layer and the length of the slits and the direction of the slits. This distance between the slits must be sufficiently great to prevent the top layer from being torn apart when the wearer moves, and sufficiently great to allow the slits to close in the desired manner under the influence of other slits, although at the same time sufficiently small for the ability to breathe and the liquid permeability to remain at an acceptable level. The durability of the top layer is largely governed, however, by the relationship between the surface containing slits and the surface without slits for a given material strength, where the distance between the slits is a subset of the parameters for the durability. The length of the slits and the distance between the slits and the direction of the slits vary depending on the material in the top layer, because the natural tendency of the slits to open depends on the characteristics of the material present in the top layer.

The backing layer is preferably liquid-impermeable (or at least possesses high resistance to penetration by liquid) and is thus so arranged as to prevent any leakage of excreted fluid from the product. The backing layer, on the other hand, may be executed so that it is vapor-permeable. For this purpose, the backing layer may be manufactured from a liquid-impermeable material which appropriately includes a thin and liquid-proof plastic film. For example, plastic films of polyethylene, polypropylene or polyester can be used for this purpose. Alternatively, a laminate of nonwoven and plastic film or other suitable layers of material can be used as a liquid-proof backing layer. In a previously disclosed manner, the under side of the backing layer can be provided with beads of adhesive or some other previously disclosed attachment means, which can then be utilized for the application of the product to an item of clothing. The product can also be provided with wings, that is to say folding flaps which, in a previously disclosed manner, are arranged along the sides of the product and can be utilized in conjunction with the application of the product.

The product can also include a further layer of material in the form of a receiving layer (also referred to as an acquisition layer, an admission layer and a distribution layer, depending on the function of the material). The receiving layer can be in the form of a wadding material having an appropriately specified thickness and resilience, which is intended to be positioned between the absorption body and the top layer. The receiving layer possesses essentially the same dimensions as the top layer, with the exception of its thickness, however, which can deviate from the thickness of the top layer. It is also possible to establish that the receiving layer can include materials other than wadding material. For example, it may include a so-called airlaid material, which is usually based on cellulose fibres. The receiving layer can also incorporate fibrous materials in order to impart an appropriately balanced rigidity to it. The receiving layer can also incorporate an appropriate quantity of thermoplastic fibres in order to permit ultrasonic welding.

The receiving layer can appropriately be a porous, elastic, relatively thick layer of material, for example in the form of a fibrous wadding material, a carded fibre wadding, a tow material, or some other kind of bulky and/or resilient fibre material with a high instantaneous liquid intake capacity that is capable of storing liquid temporarily before it is absorbed by the subjacent absorption body. The receiving layer can also be in the form of a porous foam material. It can also include two or more layers of material. According to a preferred embodiment, the receiving layer can extend towards the lateral edges of the product, that is to say it possesses essentially the same form as the top layer. In this way, advantages can be achieved in respect of liquid distribution, edge sealing, etc.

When manufacturing the absorbent product, the top layer is joined to the backing layer and can also be joined to the receiving layer and/or the absorption body. Joining can take place by gluing; or by welding in the form of ultrasonic or laser; or by mechanical joining, for example in the form of embossing or compression, etc., or by some other appropriate method of joining, for example thermal bonding.

In a preferred embodiment, at least the wet area comprises a perforated two-dimensional or three-dimensional plastic or nonwoven film, where the rest of the top layer comprises a slit, nonwoven in the lateral zones and, where appropriate, in the front section and/or in the rear section of the central zone.

The choice of material and the thickness and density of the layer of material may change in the future in the event of changed manufacturing methods and new material combinations, as a consequence of which the invention is not restricted to the materials and material combinations indicated above.

The lotion substance can be laid in accordance with the invention between the top layer and a layer lying directly under the top layer, which layer can be constituted by a backing layer or an absorption layer or a receiving layer, depending on the construction of the product and thus on which layer lies closest to the top layer. The lotion substance can thus be arranged on a backing layer or an absorption layer or a receiving layer, but it can also be laid loosely between the top layer and the immediately subjacent layer. The lotion substance is laid in that part of the product that is slit in order to enable the absorbent product, when it is being worn, to distribute lotion onto a wearer's skin via the slits.

A lotion substance according to the invention can be a skin care substance which has a positive effect on the wearer's skin and which, for example, can prevent the occurrence of skin irritation or can assist with the healing of various skin irritations. The lotion substance can also be a substance which cools the wearer's skin, for example calamine lotion, or which transfers active substances to the wearer via the wearer's skin and/or mucous membranes, or which contains odor-inhibiting substances, or which contains fragrant substances such as perfume or the like.

Suitable lotion substances can be in solid form, semi-solid form, or can be relatively liquid at ca 20° C., that is to say at room temperature. The use of slits instead of holes minimizes the risk of “leakage” of a relatively liquid lotion when the product is not in use. Lotion of a solid or semi-solid type preferably becomes liquid at the time of using the absorbent product because of the wearer's body temperature and possibly also because the lotion substance is exposed to shearing forces and frictional forces which increase the energy content of the lotion substance in such a way that a phase transition takes place from solid to liquid form, in conjunction with which the slits dispense lotion in the desired manner onto the wearer's skin. The transition is dependent, among other things, on the lotion substance comprising essentially solid components and, in addition, a small quantity of liquid components. The expression “semi-solid” denotes a substance with rheological characteristics that are typical of a pseudoplastic material or plastic fluids.

Skin care products can contain an emollient including an immobilizing substance and, where appropriate, a hydrophilic surface-active agent and other optional components. The expression emollient is used here to denote a substance which softens, soothes, relieves, makes supple, coats, lubricates, moisturizes or cleans the skin. An emollient can produce one or more of these effects. The emollient can be entirely free from water or can contain a smaller or larger quantity of water. The emollient can be petroleum-based; or it can be of the fatty acid type, or the fatty acid ester type, or the alkyl ethoxylate type, or the fatty acid ester alkyl ethoxylate type; or it can be of the fatty alcohol type, or the polysiloxane type; or a mixture of these.

Polysiloxane compounds can contain the following structure: R1-(SiO⁻)-R2, where R1 and R2, for each independent siloxane monomer unit, can be radicals in the form of hydrogen, alkyl, aryl, alkenyl, alkaryl, arakyl, cycloalkyl, halogenized hydrocarbon, or a radical. Each of these radicals can be replaced or retained. Each R1 and R2 radical can differ from the corresponding functionality of the subsequent monomer unit. The polysiloxane compound can possess structure including a straight chain or a branched chain or a cyclic chain. The R1 and R2 radicals can also contain the following functionalities, for example: alcohol, carboxylic acid, phenyl and amine functionalities. Examples of alkyl radicals are: methyl, ethyl, propyl, butyl, pentyl, hexyl, etc. Examples of alkenyls are: vinyl, allyl, etc. Examples of aryls are: phenyl, diphenyl, naphthyl, etc. R1 and R2 can also contain any suitable substance/group which gives a desired effect for the wearer. Suitable polysiloxane compounds are described in U.S. Pat. No. 5,059,282.

Examples of other suitable emollients include propylene glycol, glycerine, triethylene glycol, spermaceti or other waxes, fatty acids and fatty alcohol ethers with 12-28 carbon atoms in the fat chain, for example stearic acid, etc.

Examples of suitable immobilizing means are: cetyl alcohol; stearyl alcohol; C₁₆-C₁₈ fatty acids, for example palmitic acid, stearic acid; polyhydroxyl fatty acid esters; sorbitane esters; wax in the form of carnauba palm wax, beeswax, paraffin wax; and mixtures of these. Examples of polyhydroxyl fatty acid esters are made up of the formula R²—CO—NR¹-Z, where R¹ is hydrogen, or C₁-C₄ hydrocarbyl, 2-hydroxy ethyl, etc., and where R² is a C₅-C₃₁ hydrocarbyl group, and where Z is a polyhydroxy hydrocarbyl unit. The Z unit can be derived from a reduction of sugar in a reductive amine reaction and can contain glycityl, for example. Maize dextrose syrup and high maize fructose syrup can also be used. A further large number of previously disclosed immobilizing substances are possible, but are not listed here.

The surface-active substances can be capable of being mixed with the emollient and the immobilizing substance in such a way that a homogeneous mixture is formed. Furthermore, the surface-active substances should be mild and non-irritating for the wearer's skin. The nature of the surface-active substances should be such that they do not migrate after the lotion substance has been applied under the top layer. The surface-active substance can be hydrophilic and should be nonionactive. The quantity of hydrophilic, surface-active substance required in order to achieve improved processing and/or stability depends on the type of emollient, immobilizing substance, characteristics of the surface-active substance, etc.

Skin care compositions can include other optional components which are normally present in emollients, creams and lotions, for example the adsorption substances for urine and faeces; enzyme inhibitors; pH-regulating substances; probiotic substances such as various forms of lactobacillus and bacteria inhibitors; anti-inflammatory substances; perfumes; oils; synthetic and natural products; different forms of ether compositions; antivirus substances; steroids; antihistamines, etc.

The object of the invention is to enable a substance positioned under the top layer to be released during use. The substance that is used can be determined by the manufacturer and can include previously disclosed and hitherto undisclosed substances, and it thus does not need to be restricted to the list of examples described above. A list of suitable ingredients for skin care products can be obtained from the CTFA (Cosmetic, Toiletry and Fragrance Association), for example.

The viscosity of the skin care product should be sufficiently high at 20° C. for the lotion not to flow out from the top layer via the slits. It must be pointed out, however, that the viscosity of the skin care product according to the invention can be lower than if the top layer had not had any slits and had instead been adapted for the lotion substance to diffuse out through the material via holes in the material. The invention offers the advantage that a substance having relatively low viscosity can be stored under an essentially watertight top layer, in conjunction with which the slits in their closed position, that is to say they contain the lotion substances when the product is not being used, and in conjunction with which the lotion substance is subsequently able, when the absorbent product is used, to pass through the intermittently open slits in order, in this way, to be brought into contact with the wearer's skin.

In one embodiment of the invention, the absorbent product contains an odor inhibitor positioned under the top layer in the wet area. Odor inhibitors are previously disclosed and include, for example, a granulate of a substance which absorbs or adsorbs malodorous substances. Examples of odor inhibitors are absorbent materials in the form of zeolites or substances which oxidize the malodorous substances, etc.

According to one embodiment of the invention, the absorbent product comprises superabsorbents (SAPs) positioned under the top layer. SAPs include granules which, on contact with liquid, absorb the liquid and are transformed into a gel-like substance.

The use of slits instead of holes gives the absorbent product the advantage that the above-mentioned SAP granules do not leak out through the slits in the same way as through the holes, because the slits behave in such a way that they are caused to open and close alternately by the movement of the product when it is being worn, unlike holes which are always open.

Another advantage of the invention is that the slits are not blocked by the lotion substance, which is intended to be brought into contact with the wearer's skin via the slits as the slits open and close. Liquid is thus able to penetrate through the top layer to the subjacent absorption body, at the same time as the lotion substance passes through the slits in the opposite direction. The movement of the slits also means that air can always find its way out through the slits, which makes the top layer breathable, a feature that is particularly important in the lateral zones because such airiness reduces the risk of moisture-related skin complaints.

### BRIEF DESCRIPTION OF THE DRAWINGS

The invention is described below in conjunction with a number of Figures, in which:

FIG. 1 depicts schematically a view of the top layer of an absorbent product in accordance with the invention;

FIG. 2 depicts schematically a sectional view along the line II-II in FIG. 1;

FIG. 3 depicts schematically an enlarged view of section A in FIG. 2;

FIG. 4 depicts schematically an absorbent product according to FIGS. 1-3 when being worn against the lower abdomen of a wearer;

FIG. 5a depicts schematically a slit according to the invention; and

FIG. 5b depicts schematically a hole according to the prior art.

### DETAILED DESCRIPTION OF THE PREFERRED EMBODIMENTS

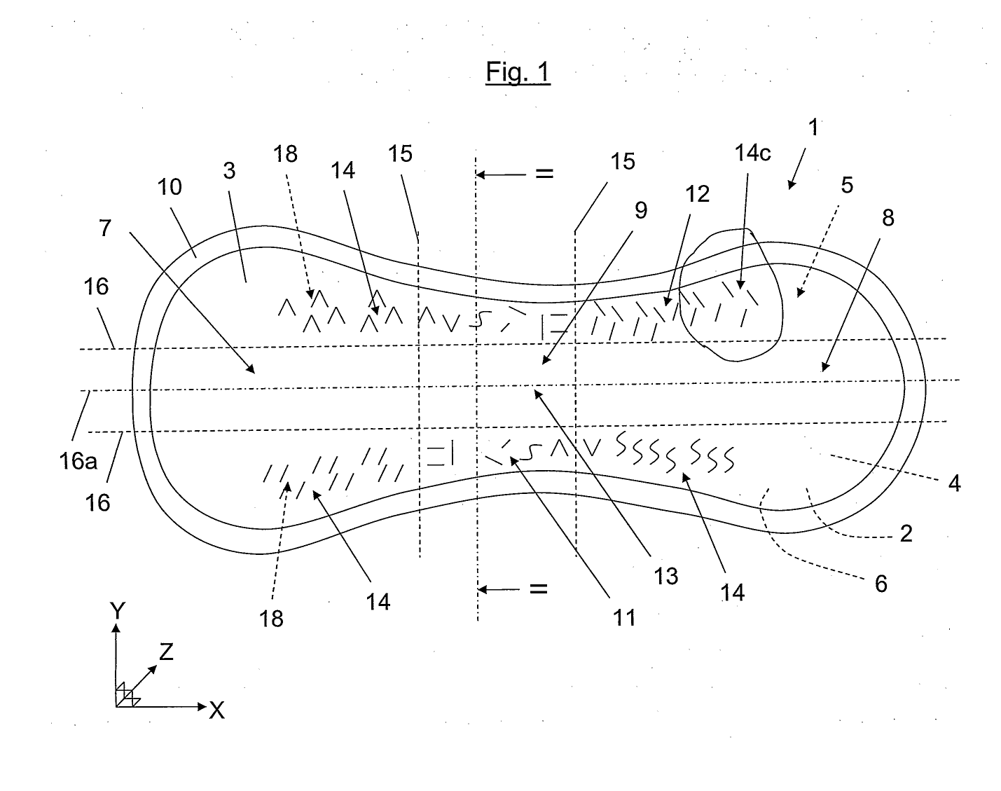

FIG. 1 depicts schematically a view of the top layer 3 of an absorbent product 1 according to the invention. The absorbent product 1 here includes a sanitary towel, which can be used as undergarment protection in conjunction with menstruation or light incontinence. The absorbent product 1 is positioned in a plane position and exhibits in this plane an extent in the longitudinal direction, the X-axis in the Figure, and in the lateral direction, the Y-axis in the Figure, and has a thickness perpendicular to the plane, the Z-axis in the Figure.

The absorbent product 1 comprises a backing layer 2, a top layer 3 and between them an absorption body 4. The absorption body 4 has a first surface 5 and a second surface 6, in conjunction with which the backing layer 2 is arranged over the second surface 6 of the absorption body 4, and the top layer 3 is arranged over the first surface 5 of the absorption body 4. The absorbent product is subdivided theoretically in the longitudinal direction into a rear section 7, a front section 8 and between them a central section 9. The absorbent product 1 is delimited by a peripheral edge part 10. The top layer 3 is described from the edge part 10, in the lateral direction, by a first lateral zone 11 and a second lateral zone 12 and positioned between them a central zone 13. The top layer 3 comprises in the lateral zones 11, 12 a layer of material containing slits 14. The slits 14 can have any desired embodiment, on condition that the slit 14 performs in the intended manner during use, that is to say the slit 14 opens and closes when the wearer moves. FIG. 1 shows a number of embodiments of slits 14, namely curve-shaped, angle-shaped, diagonal, straight, and mixtures of the various types. The various types of slits 14 are shown in the same absorbent product 1 only for the purpose of enabling the simple illustration of different types of slits 14. The invention is not restricted to comprising different types of slits 14, but can be executed with a number of similar slits 14, or with a number of optional combinations. Depicted in FIG. 1 are two laterally extending lines 15, which divide the product in the longitudinal direction in accordance with the above, and two lines 16 extending in the longitudinal direction, which divide the product in the lateral direction in accordance with the above. The direction in which the slits 14 extend differs appropriately, at least partially, from the longitudinal direction of the product and from the lateral direction of the product, because such an arrangement of the slits involves a greater degree of influence from forces acting in different directions.

Depicted in FIG. 1 is a preferred embodiment of the shape of the slits 14, where the preferred combination of slits 14 has been ringed and identified with the reference designation 14c. The slits 14c contain a combination of a plurality of straight slits 14c arranged in a row having the same or a different length, where every other slit is oriented at an angle towards the previous slit, but where the slits are situated at a distance from one another. The slits 14c are arranged at an angle of between 0° and 180° relative to a longitudinally extending centre line 16a, preferably in the range from 20°-65° and/or 110°-155° in relation to the longitudinally extending centre line 16a. The diagonally extending slits 14c provide a stronger layer of material than longitudinally extending slits or laterally extending slits for a given length of the slit.

FIG. 1 shows that a lotion substance 18 has been laid directly under the top layer 3 adjacent to the slits 14.

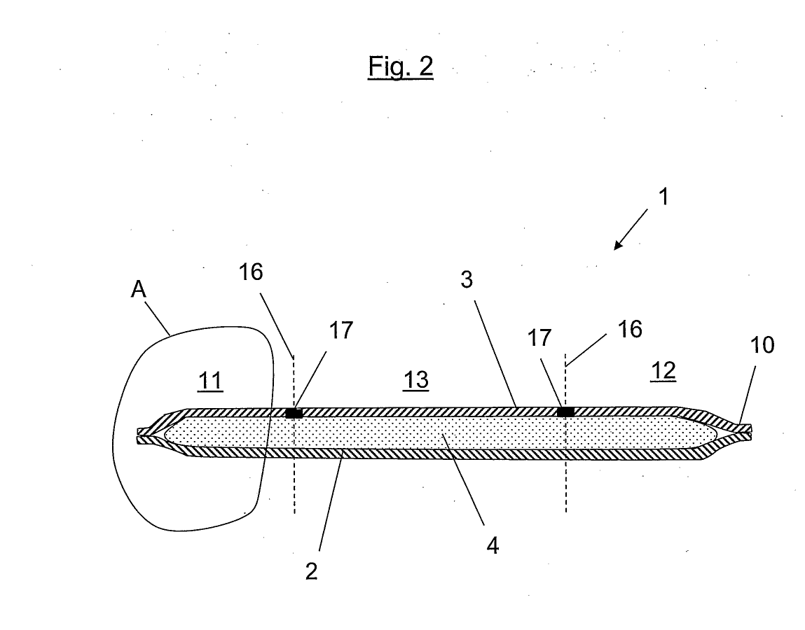

FIG. 2 depicts schematically a sectional view along the line II-II in FIG. 1. FIG. 2 shows that the top layer 3 comprises two joining sections 17 along the lines 16 which form the boundary between the lateral zones 11, 12 and the central zone 13. The joining sections 17 join the central zone with the lateral zones and can be executed with any suitable method of joining, for example welding or gluing. The joining sections 17 are used when the top layer 3 includes two different materials, that is to say when the central zone 13 includes a first material and the respective lateral zone 11, 12 includes a second material. One advantage of different materials in the different zones 11, 12, 13 is that the material in the central zone 13 can be adapted to the conditions in the central zone 13, and that the material in the lateral zones 11, 12 can be adapted to the conditions in the lateral zones 11, 12. The conditions in the central zone 13 differ considerably from the conditions in the lateral zones 11, 12 because the central zone 13 is not influenced by the wearer's body movements to the same extent as the lateral zones 11, 12. The central zone 13 also comprises the previously described wet area, and the material is accordingly adapted depending on the envisaged area of application of the product, for example as a panty liner, incontinence pad, sanitary towel or the like, because different areas of application impose different requirements on the top layer in respect of, for example, the capacity to admit liquid, the feeling of dryness, the microclimate, etc.

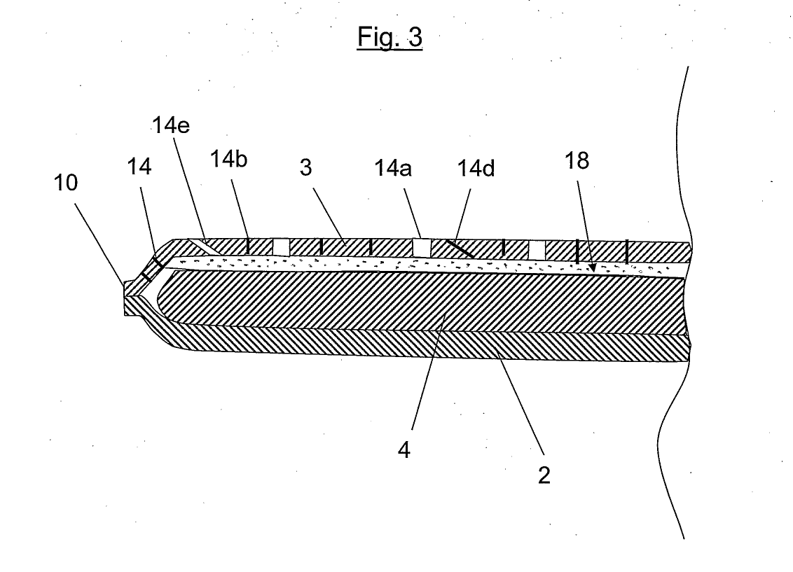

FIG. 3 depicts schematically an enlarged view of section A in FIG. 2. FIG. 3 shows that the top layer 3 contains a number of slits 14, of which a proportion 14a are open and a proportion 14b are closed. The reason why FIG. 3 shows that a proportion of slits 14a are open and a proportion of slits 14b are closed is that this symbolizes the actual condition of the top layer 3 when the lateral zones 11, 12 are influenced by the wearer's body movement when wearing the absorbent product 1. The wearer's body movement transmits a movement to the lateral zones 11, 12, which movement influences the slits 14 in such a way that they open and close. This differs from the conditions in the central zone 13, where the top layer 3 is not influenced in the same way by the wearer's body movement, but to a lesser extent, as a result of which the slits 14 do not open and close in the same way as in the lateral zones 11, 12. Depicted in FIG. 3 are slits 14b cut perpendicularly to the top layer and slits 14d cut at an angle to the top layer. In the latter case, an overlapping section is obtained in the slit, in that the boundary surfaces of the slit in the top layer through the angled slit have an extent in the Z-axis and in the X-axis and/or the Y-axis. The overlapping section provides a slightly better sealing ability than a perpendicular incision when the slit is closed. Depicted in FIG. 3 is a partially opened, inclined slit 14e, which gives a better seal against the outward passage of material lying loosely under the top layer, at the same time as the slit permits a high transport of air.

FIG. 3 also shows that the absorption body 4 comprises a lotion substance 18 laid in the lateral zones 11, 12 between the top layer 3 and the absorption body 4. The lotion substance 18 is arranged on the first surface 5 of the absorption body 4, but it can also be laid separately between the top layer 3 and the absorption body 4 in conjunction with manufacture of the absorbent product 1. Lotion substance 18 can also be laid along the edge part 10 of the absorbent product 1 in the space formed between the top layer 3, the backing layer 2 and the absorption body 4.

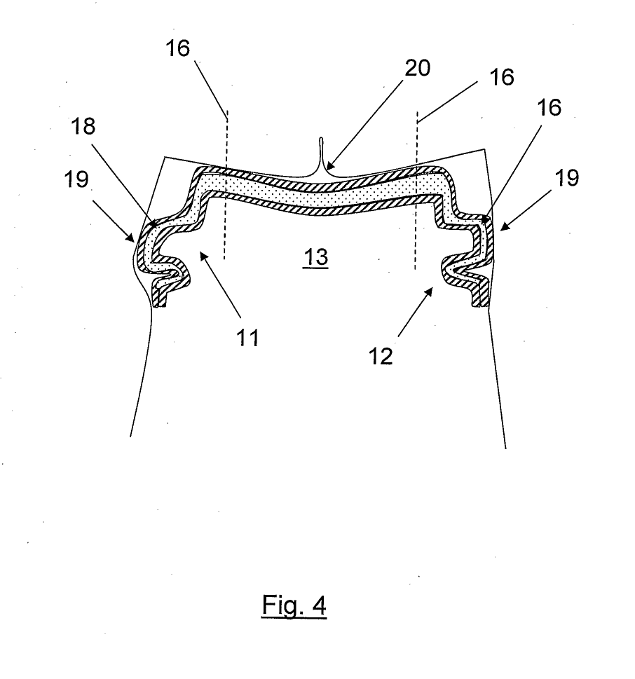

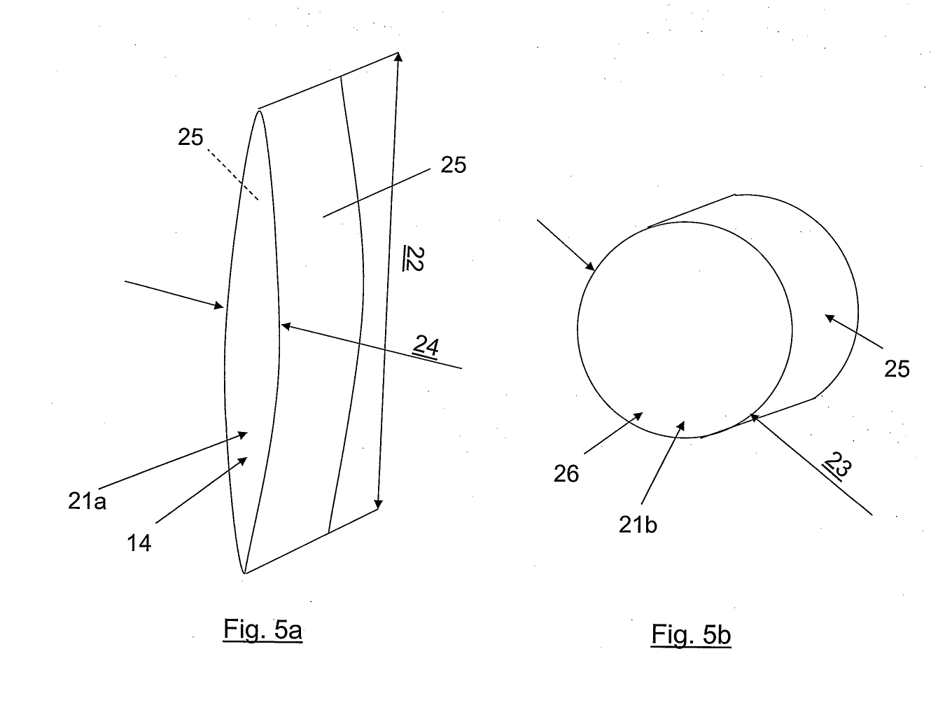

FIG. 4 depicts schematically an absorbent product 1 according to FIGS. 1-3 positioned between the inside of the thighs 19 and against the lower abdomen 20 of a wearer. FIG. 4 depicts how the absorbent product 1 has been deformed by the lateral zones 11, 12 having been folded down in relation to the central zone 13, which is attributable to the fact that the absorbent product 1 has a greater extent than the relatively plane part of the wearer's lower abdomen. FIG. 4 also shows that the lateral zones 11, 12 have been deformed by the lateral zones 11, 12 having been crumpled as a result of the wearer's leg movements. It is evident from FIG. 4 that the central zone 13 has a more static environment than the lateral zones 11, 12. The expression static is used here to denote that the central zone 13 is less exposed to movement than the lateral zones 11, 12. The problems at the lateral zones 11, 12 referred to above thus do not arise in the same way at the central zone 13, and above all not in the central section 9, for which reason the central zone 13 of the top layer can include a different material than the lateral zones 11, 12 of the top layer. The lateral zones 11, 12 can be adapted in such a way that the wearer experiences the material as comfortable, which is the case with a soft material which possesses a dry feel even after the wearer has excreted fluid. The material in the lateral zones 11, 12 can be water-permeable with low reflux, furthermore, and it can permit lotion 18 to find its way out through the top layer 3 when the wearer moves. This is achieved through the invention because the lateral zones 11, 12 of the top layer 3 are embodied with slits 14 which open and close during movement. The difference between using slits and holes is illustrated in FIGS. 5a and 5b below.

FIG. 5a depicts schematically a slit 14 according to the invention, and FIG. 5b depicts schematically a hole 26 according to the prior art. The slit 14 is depicted in FIG. 5a in an open position (14a in FIG. 3), where the slit 14 comprises an opening 21a and where the hole 26 comprises a corresponding opening 21b. The slit 14 advantageously has a length 22 which exceeds the diameter 23 of the hole 26, in conjunction with which the opening 21a in the slit 14 can have an area larger than that of the hole 26. It is possible for the width 24 of the opening to exceed the diameter 23 of the hole during a short period of time, although because the slit 14 moves between an open and closed position, lotion is dispensed through the slit in a suitable dose. When the absorbent product is not being used, the opening 21a in the slit 14 is closed, although the opening 21b in the hole 26 nevertheless always remains open regardless of whether or not the product is being worn. The slit 14 is shown in FIG. 5a to comprise two boundary surfaces 25, each delimiting the extent of the slit 14 in the longitudinal direction 22 and the thickness of the layer of material.

FIG. 5b depicts schematically a hole 26 according to the prior art, in which the opening 21b includes a hole 26 contained in the top layer 3. The hole 26 is formed by material in the top layer 3 having been removed. One effect of having removed material is that the hole 26 is always open, regardless of whether the absorbent product is being worn. The hole 26 is certainly capable of being deformed under external influence, although there is very little likelihood of the hole 26 being deformed in such a way that the opening 21b is closed or becomes so small that lotion is not able to find its way out. If the lateral zones 11, 12 in the top layer were to be provided with holes 26 instead of slits 14, the holes 26 would thus always be open regardless of the movements of the wearer and the associated movements of the lateral zones 11, 12, in conjunction with which the previously mentioned problems would present themselves. The slit described in FIG. 5a thus possesses all the advantages mentioned above compared with the hole depicted in FIG. 5b.

1. An absorbent product having an extent in a plane in the longitudinal direction and the lateral direction and a thickness perpendicular to the plane when the product is positioned in a plane position, the absorbent product comprising a backing layer and a top layer, the absorbent product including in the longitudinal direction a rear section, a front section and positioned between them a central section, and including in the lateral direction a first lateral zone, a second lateral zone and positioned between them a central zone, wherein the top layer comprises slits as the only openings in an area which fully or partially encloses that part of the absorbent product that is formed by the section of the central zone and the central section, which constitutes the wet area of the product, and wherein lotion is placed directly under the top layer within the area containing slits.

2. The absorbent product according to claim 1, wherein the slits are arranged in one or both lateral zones.

3. The absorbent product according to claim 1 wherein the slits are arranged in the central section of one or both lateral zones.

4. The absorbent product according to claim 1, wherein the slits are arranged only in one or both lateral zones.

5. The absorbent product according to claim 4, wherein the slits are arranged only in the central section of one or both lateral zones.

6. The absorbent product according to claim 1, wherein the slits are arranged in the front section and/or in the rear section.

7. The absorbent product according to claim 1, wherein the slits have a length of 2-15 mm.

8. The absorbent product according to claim 1, wherein the slits are arranged fully or partially at an angle of between 0° and 180° relative to a centre line through the product extending in the longitudinal direction.

9. The absorbent product according to claim 1, wherein the slits are straight, S-shaped, V-shaped, Z-shaped, or U-shaped.

10. The absorbent product according to claim 1, wherein that the slits contain a combination of a plurality of straight slits arranged consecutively having the same or a different length, where every other slit is oriented at an angle to the previous slit, but where the slits are situated at a distance from one another.

11. The absorbent product according to claim 1, wherein one or a plurality of slits are made in the top layer through a through going incision in the top layer perpendicular to the plane.

12. The absorbent product according to claim 1, wherein one or a plurality of slits are made in the top layer through a through going incision in the top layer at an angle to the plane.

13. The absorbent product according to claim 1, wherein the section of the central zone and the central section comprises at least one perforated two-dimensional or three-dimensional plastic or nonwoven film, and wherein the rest of the top layer comprises a slit, nonwoven in at least a part of the lateral zones and, optionally, in the front section and/or in the rear section.

14. The absorbent product according to claim 1, wherein the absorbent product comprises an absorption body positioned between the top layer and the backing layer, the absorption body having a first surface and a second surface where the top layer is arranged over the first surface of the absorption body and the backing layer is arranged over the second surface of the absorption body.

15. The absorbent product according to claim 14, wherein the absorbent product comprises a receiving layer positioned between the top layer and the absorption body.

16. The absorbent product according to claim 7, wherein the slits have a length of 3-10 mm.

17. The absorbent product according to claim 8, wherein at least some of the slits are arranged at an angle in the range from 20°-65° in relation to the centre line extending in the longitudinal direction.

18. An absorbent product according to claim 8, wherein at least some of the slits are arranged at an angle in the range from 110°-155° in relation to the centre line extending in the longitudinal direction.
